(19) United States
(12) Patent Application Publication
(10) Pub. No.: US 2012/0083622 A1
(43) Pub. Date: Apr. 5, 2012

(54) METHOD AND APPARATUS FOR PREPARING AN ESTER

(75) Inventors: HSIAO-PING HUANG, Taipei (TW); I-LUNG CHIEN, Taipei (TW); MING-JER LEE, Taipei (TW); I-KUAN LAI, Taipei (TW)

(73) Assignee: National Taiwan University, Taipei (TW)

(21) Appl. No.: 12/940,344

(22) Filed: Nov. 5, 2010

(30) Foreign Application Priority Data
Oct. 4, 2010 (TW) 099133777

(51) Int. Cl. C07C 67/60; B01J 19/00; B01D 3/00; B01J 8/02

(52) U.S. Cl. 560/248; 202/153

(57) ABSTRACT

A method for preparing an ester is provided. The method includes steps of mixing an acid and an alcohol in a reactive distillation column to generate a first gas mixture; transporting the first gas mixture out of the reactive distillation column; cooling down the first gas mixture for a phase separation to obtain a first liquid mixture in an upper phase; transporting the first liquid mixture back to the reactive distillation column; obtaining a second liquid mixture at a middle section of the reactive distillation column; transporting the second liquid mixture to a separative distillation column; and obtaining the ester at a bottom section of the separative distillation column.

### FIELD OF THE INVENTION

The present invention relates to a method and, an apparatus for preparing an ester, especially to a method and an apparatus for preparing an ester with high purity of the industrial grade.

### BACKGROUND OF THE INVENTION

Ethyl acetates and isopropyl acetates are solvents of great importance in the industry, which are commonly utilized for plastics, oils, printing inks and materials for synthesized perfumes. Nowadays, the processes for producing the ethyl acetate could be typically classified as follows:

(1) The process proposed by Keyes (1932) involves an esterification reaction of acetate in a pre-reactor and plural purification and recycling procedures of the ester product in plural strippers and phase separation tanks. Such process, however, is relatively complicated owing to more reaction units involved therein, and is hence disadvantageous in system operation and maintenance.

(2) There are plenty of researches studying the single reactive distillation (RD) column designed process. Such process is carried out with a typical RD column, which is principally divided into three sections including the reaction section, the purification section and the stripping section, and therein the esterification reaction is promoted with a homogeneous or a non-homogenous catalyst. The single RD column designed process for ethyl acetate is firstly proposed by Suzuki et al. in 1972 (Vora and Daoutidis, 2001), where the operation and control of the RD column for an ethyl acetate system is investigated. Nevertheless, in such a process, the purity of the ester product obtained at the top section of RD column is not high enough, and the product obtained at the bottom section of RD column, containing four different ingredients, still needs to be further processed. The data in those researches show that the ethyl acetate product obtained in such an RD column has a relatively low purity, and thus fails to meet the current industrial demands. Typically, the product obtained at the top section of such RD column is restricted by the acid-alcohol-ester azeotrope, and the product obtained at the bottom section thereof is restricted by the high boiling point of acetate. Accordingly, it may need to discharge or further process the undesired product. As disclosed in U.S. Pat. No. 6,693,213, for example, on one hand the organic product obtained at the top section of RD column needs to be separated and is then discharged therefrom, and on the other hand, the acid reactant with a high purity needs to be recycled at the bottom section of RD column, both bringing disadvantageous in the esterification process and the energy consumption.

(3) The process proposed by Bock (1997) is based on a design of two distillation columns, including an RD column and a stripper. In this case, it is possible to obtain the ethyl acetate with a relatively high purity at the bottom of the second column, i.e. the stripper, while the mixture of acid-alcohol-ester azeotrope obtained at the top thereof needs to be transported back to the first column, i.e. the RD column, to be processed, and the mixture of alcohol and water, that is obtained at the bottom of RD column, also needs to be further treated.

For overcoming the mentioned drawbacks existing in the conventional techniques, a novel method and apparatus for the esterification is provided in the present invention with the advantages of saving more energies and producing the acetate of higher purity.

### SUMMARY OF THE INVENTION

The present invention provides the method and the apparatus for preparing an ester to obtain the acetate with the industrial level of high purity.

In accordance with one aspect of the present invention, a method for preparing an ester is provided. The method comprises steps of mixing an acid and an alcohol in a reactive distillation column to generate a first gas mixture; transporting the first gas mixture out of the reactive distillation column; cooling down the first gas mixture for performing a phase separation to obtain a first liquid mixture in an upper phase; transporting the first liquid mixture back to the reactive distillation column; obtaining a second liquid mixture at a middle section of the reactive distillation column; transporting the second liquid mixture to a separative distillation column; and obtaining the ester at a bottom section of the separative distillation column.

In accordance with another aspect of the present invention, an apparatus for preparing an ester is provided. The apparatus comprises a reactive distillation column having a middle section, and fed with an acid and an alcohol for a reaction to generate a first gas mixture; a phase separation tank inputted with the first gas mixture for a phase separation to generate a first liquid mixture, and transporting the first liquid mixture back to the reactive distillation column; and a separative distillation column having a bottom section, and inputted with a second liquid mixture from the middle section of the reactive distillation column for a distillation separation, wherein the ester is obtain at the bottom section of the separative distillation column,

The above objects and advantages of the present invention will become more readily apparent to those ordinarily skilled in the art after reviewing the following detailed descriptions and accompanying drawings, in which:

### BRIEF DESCRIPTION OF THE DRAWINGS

FIG. 1 is the schematic diagram showing an apparatus for manufacturing an ester in one embodiment of the present invention.

### DETAILED DESCRIPTION OF THE PREFERRED EMBODIMENT

The present invention will now be described more specifically with reference to the following embodiments. It is to be noted that the following descriptions of preferred embodiments of this invention are presented herein for the purposes of illustration and description only; it is not intended to be exhaustive or to be limited to the precise form disclosed.

According to the present invention, the apparatus for producing the ester is aimed at the design of the thermal integration for the reactive distillation (RD) column of the esterification so as to obtain the ester with the high purity and the energy saving. The esterification is typically shown as follows:

acid+alcohol [symbol] acetate+water

The esterification is a reversible reaction, wherein the acetate may be ethyl acetate, isopropyl acetate, n-propyl acetate or butyl acetate, the acid may be acetic acid, and the alcohol may be ethanol, isopropanol, n-propane or butanol. The catalyst typically used in this reaction may be a homogeneous or a non-homogeneous catalyst. Preferably, the non-homogeneous catalyst, i.e. a solid catalyst, is adopted in the RD column according to the present invention. Since the solid catalyst could be arranged in any section, e.g. the bottom section and the top section, of the RD column by means of filling, it is able to flexibly select any desired section of the RD column for the reaction. Moreover, the issue of the difficult recycling procedures for the liquid catalyst can be accordingly avoided. Typically, the ionic exchange resin is adoptable as the solid catalyst, e.g. Amberlyst 15 (Rohm and Hass) or Purolite CT179 (Purolite). The filling structures of the catalyst can be done by Katapak-S method or by filling the catalyst in a fixture arranged inside the column (Davy Process Technology).

Firstly, regarding the physical properties of the reaction system for ethyl acetate generated by reacting the acetic acid with the ethanol, the boiling points of the respective ingredients involved in the ethyl acetate system are listed in the following TABLE 1.

TABLE 1

| Ingredients | Boiling Point (° C.) |
|---|---|
| EtOH/EtAc/H₂O | 70.09 |
| EtAc/H₂O | 70.37 |
| EtOH/EtAc | 71.81 |
| EtAc | 77.20 |
| EtOH/H₂O | 78.18 |
| EtOH | 78.31 |
| H₂O | 100.0 |
| HAc | 118.01 |

The azeotrope ingredients include the alcohol-ester-water azeotrope and the acetate-water, the alcohol-ester and the alcohol-water, azeotropes, where the existence of azeotropes brings a limitation to separation on the distillation boundary. Among the mentioned ingredients, the mixture of alcohol-ester-water azeotrope (EtOH/EtAc/H₂O) has a lowest boiling point of 70.09° C. and is hence regarded as a light ingredient, while the heavy ingredient, acetic acid (HAc), has a highest boiling point of 118.01° C. In a typical RD column, the mixture obtained at the top thereof has a composition approaching to the alcohol-ester-water azeotrope, and the product obtained at the bottom thereof is acetic acid. The desired product, ethyl acetate, has a boiling point of 77.2° C., which is ranged between those of the respective alcohol-ester-water azeotrope and acetic acid, and this is why the product obtained from the conventional single RD column would has a relatively low purity.

Furthermore, the boiling points of the respective ingredients involved in the isopropyl acetate system are listed in the following TABLE 2. It is apparent that the esterification system for isopropyl acetate has the same limitation in distillation as that for ethyl acetate, and accordingly the desired product with a middle-ranged boiling point is difficult to be obtained therefrom.

TABLE 2

| Ingredients | Boiling Point (° C.) |
|---|---|
| IPA/IPAc/H₂O | 74.22 |
| IPAc/H₂O | 76.57 |
| IPA/IPAc | 78.54 |
| IPA/H₂O | 80.06 |
| IPA | 82.35 |
| IPAc | 88.52 |
| H₂O | 100.0 |
| HAc | 118.01 |

The apparatus for esterification according to the present invention is constructed by an RD column, a phase separation tank and a separative distillation column (i.e. stripper), where the RD column is only divided into a purification section and a reaction section. The reaction section is referred to the section in which the trays are filled with solid catalysts, and is arranged in a region from a middle section (or a middle-to-upper section) to a bottom section of the RD column. The purification section is arranged above the reaction section. The reactants, including the acetic acid and the alcohol, are fed into the RD column at the bottom section thereof, or fed through a re-boiler. Since the large amount of the acid with high purity would stay at the bottom section of the RD column, it needs to fill the catalyst at the bottom section with a filling amount larger than 10-100 times of that in the other reaction section so as to facilitate the reaction of the acetic acid and alcohol. The input (feed) quantity of the alcohol into the RD column is somewhat larger than that of the acid, in an acid-to-alcohol molar ratio around 1:1.05 and ranged from 1.0:1.0 to 1.0:1.1, preferably. The mixture with the major composition of three-ingredient and two-ingredient azeotropes is condensed in the phase separation tank so as to separate the organic phase of the products from the water phase thereof by applying the rule of the insolubility between the organic solution and the aqueous solution.

In the mentioned aspect, the present invention provides a complete reactive distillation processes for the esterification reaction of ethyl acetate and isopropyl acetate. The apparatus for carrying such processes includes an RD column, a phase separation tank and a separative distillation column (i.e. stripper). The RD column is divided into a purification section and a reaction section, and the reactants are fed into the RD column at the bottom section thereof. The reaction section of the RD column is filled with the solid catalyst. The mixture of alcohol-ester-water azeotrope obtained at the top section of RD column is transported into the phase separation tank for the phase separation. Then the organic phase solution thereof is refluxed to the top section of RD column; while the water phase liquid is outputted as a by-product, water. The liquid mixture generated at the middle section of the RD column is transported the top section of the stripper, and the ester product with relatively high purity can be obtained at the bottom of stripper.

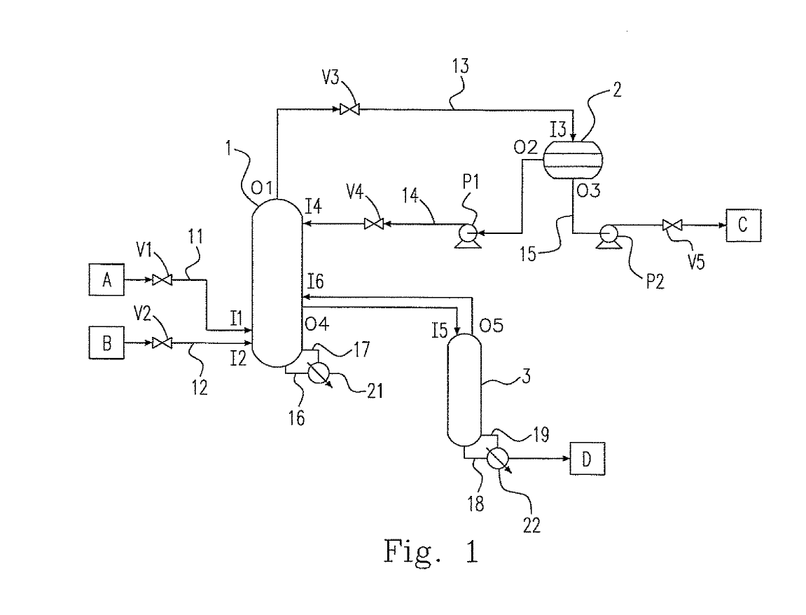

Please refer to FIG. 1, which is the schematic diagram shows an apparatus for manufacturing an ester in one embodiment of the present invention. The processes of the method for manufacturing the ester according to the present invention are illustrated based on FIG. 1 as follows.

The apparatus for manufacturing the ester in the present embodiment mainly includes a reactive distillation column 1, a phase separation tank 2 and a separative distillation column (i.e. stripper) 3. This apparatus can further include a first supply tank A for storing an acid (e.g. acetic acid) and a second supply tank B for storing an alcohol (e.g. ethanol). The apparatus for manufacturing an ester in this embodiment can include a first pipe 11, which connects the first supply tank A and the bottom section of the reactive distillation column 1, and can be configured with a first valve V1 so as to control the switch-on and switch-off for the acid feeding. The acid can be transported through the first pipe 11, via the first valve V1, to the first input port 11 at the bottom section of the reactive distillation column 1. Similarly, the apparatus for manufacturing an ester in this embodiment can include a second pipe 12, which connects the second supply tank B and the bottom section of the reactive distillation column 1, and can be configured with a second valve V2 so as to control the switch-on and switch-off for the alcohol feeding. The alcohol can be transported through the second pipe 12, via the second valve V2, to the second input port 12 at the bottom section of the reactive distillation column 1.

The acid and the alcohol inside the reactive distillation column 1 are reacted with each other as the so-called esterification reaction under the catalytic aid of the catalyst. Then, the first gas mixture, including the alcohol-ester-water, the ester-water, and alcohol-ester azeotropes, is generated at the top section of the reactive distillation column 1. The apparatus for manufacturing an ester in this embodiment can include a third pipe 13, which connects the reactive distillation column 1 and the phase separation tank 2, and can be configured with a third valve V3. The first gas mixture can flow from the first output port O1 at the top section of the reactive distillation column 1 into the third pipe 13, via the third valve V3, and finally into the phase separation tank 2 for the phase separation. In the present invention, the temperature control range for the reactive distillation column 1 can be selected and determined based on the product system. As the example of the ethyl acetate, the temperature of the reactive distillation column 1 is controlled in a range of 60 to 130° C.; while the temperature of the top section is controlled in a range of 60 to 80° C., and that of the bottom section is controlled in a range of 110 to 130° C. The temperature of the phase separation tank 2 is controlled in a range of 40 to 50° C.

The apparatus for manufacturing an ester in this embodiment can include a fourth pipe 14, which connects the reactive distillation column 1 and the phase separation tank 2, can be configured with a first pump P1 and a fourth valve V4. The first gas mixture is condensed in the phase separation tank 2 for the phase separation. The first liquid mixture is generated in the upper organic phase; while the water is generated in the lower water phase, and the water is the by-product of the esterification reaction in the present invention. The phase separation tank 2 includes a second output port O2 so that the first liquid mixture in the upper phase can flow from the output port O2, into the fourth pipe 14, via the first pump P1, the fourth valve V4 and the fourth input port 14 at the top section of the reactive distillation column 1, and finally back into the reactive distillation column 1 to continue to participate in the esterification reaction.

The apparatus for manufacturing an ester in this embodiment can include a fifth pipe 15, which connects the phase separation tank 2 and the by-product tank C, and can be configured with a second pump P2 and a fifth valve V5. The water in the lower phase of the phase separation tank 2 can flow from the third output port O3 at the bottom section of the phase separation tank 2, into the fifth pipe 15, via the second pump P2 and the fifth valve V5, and finally into the by-product tank C.

There exists a fourth output port O4 at the middle section of the reactive distillation column 1. The second liquid mixture generated at the middle section of the reactive distillation column 1 can flow from the fourth output port O4, via the fifth input port 15 of the separative distillation column (i.e. stripper) 3, and finally into the separative distillation column 3 for the distillation separation. The second gas mixture generated at the top section of the separative distillation column 3 can flow from the fifth output port O5 at the top section of the separative distillation column 3, via the sixth input port 16 at the middle section of the reactive distillation column 1, and finally back into the reactive distillation column 1 to continue to participate in the esterification reaction. In the present invention, the temperature control range for the separative distillation column 3 can be selected and determined based on the product system. As the example of the ethyl acetate, the temperature of the top section of the separative distillation column 3 is controlled in a range of 55 to 65° C., and that of the bottom section of the separative distillation column 3 is controlled in a range of 75 to 85° C.

The apparatus for manufacturing an ester in this embodiment can include a sixth pipe 16 and a seventh pipe 17, each of which connects the reactive distillation column 1 and the first reboiler 21. The third liquid mixture at the bottom of the reactive distillation column 1 flows through the sixth pipe 16 into the first reboiler 21 for the heating, and then flows through the seventh pipe 17 back into the reactive distillation column 1.

The apparatus for manufacturing an ester in this embodiment can include an eighth pipe 18 and a ninth pipe 19, each of which connects the separative distillation column 3 and the second reboiler 22. The fourth liquid mixture at the bottom of the separative distillation column 3 flows through the eighth pipe 18 into the second reboiler 22 for the heating. Then, a portion of the heated fourth liquid mixture in the second reboiler 22 flows through the ninth pipe 19 back into the reactive distillation column 1, and the other portion of the heated fourth liquid mixture in the second reboiler 22 is outputted to the product tank D so as to obtain the product, i.e. ester.

The examples of the present invention are described in the followings.

In a first example of the present invention, a method for preparing an ester is provided. The method comprises steps of mixing an acid and an alcohol in a reactive distillation column to generate a first gas mixture; transporting the first gas mixture out of the reactive distillation column; cooling down the first gas mixture for performing a phase separation to obtain a first liquid mixture in an upper phase; transporting the first liquid mixture back to the reactive distillation column; obtaining a second liquid mixture at a middle section of the reactive distillation column; transporting the second liquid mixture to a separative distillation column; and obtaining the ester at a bottom section of the separative distillation column.

In a second example of the present invention, a method of the first example further comprises a step of introducing a catalyst into the reactive distillation column for accelerating the preparation of the ester, wherein the catalyst includes an ionic exchange resin.

In a third example of the present invention, a method of any one of the preceding examples further comprises steps of obtaining a second gas mixture at a top section of the separative distillation column; and transporting the second gas mixture generated back to the reactive distillation column.

In a fourth example of the present invention, a method of any one of the preceding examples further comprises steps of obtaining a third liquid mixture at the bottom section of the reactive distillation column; transporting the third liquid mixture to a first reboiler for heating the third liquid mixture; and transporting the third liquid mixture from the first reboiler back to the bottom section of the reactive distillation column.

In a fifth example of the present invention, a method of any one of the preceding examples further comprises steps of obtaining a fourth liquid mixture generated at a bottom of the separative distillation column; transporting the fourth liquid mixture to a second reboiler for heating the fourth liquid mixture; transporting a first portion of the fourth liquid mixture from the second reboiler back to the separative distillation column; and outputting a second portion of the fourth liquid mixture from the second reboiler to obtain the ester.

In a sixth example of the present invention, the reactive distillation column in a method of any one of the preceding examples has a first temperature controlled in a range of 60 to 130° C., the reactive distillation column in a method of any one of the preceding examples has a bottom section having a second temperature controlled in a range of 110 to 130° C., the reactive distillation column in a method of any one of the preceding examples has a top section having a third temperature controlled in a range of 60 to 80° C., and the reactive distillation column a method of any one of the preceding examples has a pressure controlled in a range of 1.0 to 1.2 atmosphere.

In a seventh example of the present invention, the acid in a method of any one of the preceding examples has a first input molar quantity, the alcohol has a second input molar quantity, and a molar ratio of the first input molar quantity to the second input molar quantity is in a range of 1.0:1.0 to 1.0:1.1.

In an eighth example of the present invention, the phase separation in a method of any one of the preceding examples is performed in a phase separation tank having a temperature controlled in a range of 40 to 50° C.

In a ninth example of the present invention, an apparatus for preparing an ester is provided. The apparatus comprises a reactive distillation column having a middle section, and fed with an acid and an alcohol for a reaction to generate a first gas mixture; a phase separation tank performing a phase separation for the first gas mixture to generate a first liquid mixture, and transporting the first liquid mixture back to the reactive distillation column; and a separative distillation column having a bottom section, and inputted with a second liquid mixture from the middle section of the reactive distillation column for performing a distillation separation therefor, wherein the ester is obtained at the bottom section of the separative distillation column.

In a tenth example of the present invention, the reactive distillation column in an apparatus of any one of the preceding examples has a bottom section and includes a first input port disposed at the bottom section of the reactive distillation column; and a second input port disposed at the bottom section of the reactive distillation column, and the apparatus of any one of the preceding examples further includes a first supply tank storing the acid; a second supply tank storing the alcohol; a first pipe connecting the first supply tank and the first input port; a second pipe connecting the second supply tank and the second input port; a first valve configured in the first pipe; and a second valve configured in the second pipe, wherein the acid is transported from the first supply tank into the first pipe, via the first valve and the first input port, and then into the reactive distillation column, and the alcohol is transported from the second supply tank into the second pipe, via the second valve and the second input port, and then into the reactive distillation column.

In a eleventh example of the present invention, an apparatus of any one of the preceding examples further comprises a catalyst disposed inside the reactive distillation column for accelerating the reaction, wherein the catalyst includes an ionic exchange resin, and the reaction includes an esterification reaction.

In a twelfth example of the present invention, the acid in an apparatus of any one of the preceding examples includes an acetic acid, the alcohol in an apparatus of any one of the preceding examples includes one selected from a group consisting of an ethanol, an n-propanol, an isopropanol and a butanol, the acid in an apparatus of any one of the preceding examples has a first input molar quantity, the alcohol in an apparatus of any one of the preceding examples has a second input molar quantity, the apparatus of any one of the preceding examples has a molar ratio of the first input molar quantity to the second input molar quantity, and the molar ratio is in a range of 1.0:1.0 to 1.0:1.1.

In a thirteenth example of the present invention, the reactive distillation column in an apparatus of any one of the preceding examples has a top section and includes a first output port disposed at the top section of the reactive distillation column; and a fourth input port disposed at the top section of the reactive distillation column; the phase separation tank in an apparatus of any one of the preceding examples has a bottom section and includes a second output port; and a third input port; and the apparatus of any one of the preceding examples further includes a third pipe connecting the first output port and the third input port; and a fourth pipe connecting the second output port and the fourth input port, wherein the first gas mixture is transported from the first output port, into the third pipe, via the third input port, and into the phase separation tank, the first liquid mixture is transported from the second output port, into the fourth pipe, via the fourth input, and into the reactive distillation column, and the apparatus has a by-product obtained at the bottom section of the phase separation tank.

In a fourteenth example of the present invention, an apparatus of any one of the preceding examples further comprises a first pump, a third valve and a fourth valve, wherein the first gas mixture is transported from the first output port, into the third pipe, via the third valve and the third input port, and into the phase separation tank, and the first liquid mixture is transported from the second output port, into the fourth pipe, via the first pump, the fourth valve and the fourth input port, and then into the reactive distillation column.

In a fifteenth example of the present invention, the phase separation tank in an apparatus of any one of the preceding examples has a bottom section, and the apparatus of any one of the preceding examples further comprises a third, output port disposed in the bottom section of the phase separation tank; a by-product tank; a fifth pipe connecting the third output port and the by-product tank; a second pump configured in the fifty pipe; and a fifth valve configured in the fifty pipe, wherein the by-product is transported from the third output port, into the fifth pipe, via the second pump and the fifth valve, and then into the by-product tank, and the by-product is water.

In a sixteenth example of the present invention, the reactive distillation column in an apparatus of any one of the preceding examples includes a fourth output port disposed at the middle section of the reactive distillation column; and a sixth input port disposed at the middle section of the reactive distillation column; and the separative distillation column in an apparatus of any one of the preceding examples has a top section and includes a fifth input port disposed at the top section of the separative distillation column; a fifth output port disposed at the top section of the separative distillation column; and a second gas mixture generated at the top section of the separative distillation column, and transported from the fifth output port, via the sixth input port, and into the reactive distillation column, wherein the second liquid mixture is transported from the fourth output port, via the fifth input port, and then into the separative distillation column.

In a seventeenth example of the present invention, the reactive distillation column in an apparatus of any one of the preceding examples has a bottom section, and the apparatus of any one of the preceding examples further comprises a first reboiler; a sixth pipe connecting the first reboiler and the bottom section of the reactive distillation column; a seventh pipe connecting the first reboiler and the bottom section of the reactive distillation column; and a third liquid mixture generated at the bottom section of the reactive distillation column, transported to the first reboiler through the sixth pipe, and then transported from the first reboiler back to the reactive distillation column through the seventh pipe.

In an eighteenth example of the present invention, An apparatus of any one of the preceding examples further comprises a second reboiler; a product tank; an eighth pipe connecting the second reboiler and the bottom section of the separative distillation column; a ninth pipe connecting the second reboiler and the bottom section of the separative distillation column; and a fourth liquid mixture generated at the bottom section of the separative distillation column, transported to the second reboiler through the eighth pipe, and includes a first portion transported from the second reboiler back to the separative distillation column through the ninth pipe; and a second portion outputted from the second reboiler to the product tank.

In a nineteenth example of the present invention, the reactive distillation column in an apparatus of any one of the preceding examples has a first temperature controlled in a range of 60 to 130° C.; a bottom section has a second temperature controlled in a range of 110 to 130° C.; and a top section has a third temperature controlled in a range of 60 to 80° C.

In a twentieth example of the present invention, an apparatus of claim 9, wherein the phase separation tank has a fourth temperature controlled in a range of 40 to 50° C., the separative distillation column has a top section having a fifth temperature controlled in a range of 55 to 65° C., and the bottom section of the separative distillation column has a sixth temperature controlled in a range of 75 to 85° C.

While the invention has been described in terms of what is presently considered to be the most practical and preferred embodiments, it is to be understood that the invention needs not be limited to the disclosed embodiments. On the contrary, it is intended to cover various modifications and similar arrangements included within the spirit and scope of the appended claims which are to be accorded with the broadest interpretation so as to encompass all such modifications and similar structures.

What is claimed is:

1. A method for preparing an ester, comprising steps of:
mixing an acid and an alcohol in a reactive distillation column to generate a first gas mixture;
transporting the first gas mixture out of the reactive distillation column;
cooling down the first gas mixture for performing a phase separation to obtain a first liquid mixture in an upper phase;
transporting the first liquid mixture back to the reactive distillation column;
obtaining a second liquid mixture at a middle section of the reactive distillation column;
transporting the second liquid mixture to a separative distillation column; and
obtaining the ester at a bottom section of the separative distillation column.

2. A method of claim 1, further comprising a step of introducing a catalyst into the reactive distillation column for accelerating the preparation of the ester, wherein the catalyst includes an ionic exchange resin.

3. A method of claim 1, further comprising steps of
obtaining a second gas mixture at a top section of the separative distillation column; and
transporting the second gas mixture generated back to the reactive distillation column.

4. A method of claim 3, further comprising steps of:
obtaining a third liquid mixture at the bottom section of the reactive distillation column;
transporting the third liquid mixture to a first reboiler for heating the third liquid mixture; and
transporting the third liquid mixture from the first reboiler back to the bottom section of the reactive distillation column.

5. A method of claim 4, further comprising steps of:
obtaining a fourth liquid mixture generated at a bottom of the separative distillation column;
transporting the fourth liquid mixture to a second reboiler for heating the fourth liquid mixture;
transporting a first portion of the fourth liquid mixture from the second reboiler back to the separative distillation column; and
outputting a second portion of the fourth liquid mixture from the second reboiler to obtain the ester.

6. A method of claim 1, wherein the reactive distillation column has a first temperature controlled in a range of 60 to 130° C., the reactive distillation column has a bottom section having a second temperature controlled in a range of 110 to 130° C., the reactive distillation column has a top section having a third temperature controlled in a range of 60 to 80° C., and the reactive distillation column has a pressure controlled in a range of 1.0 to 1.2 atmosphere.

7. A method of claim 1, wherein the acid has a first input molar quantity, the alcohol has a second input molar quantity, and a molar ratio of the first input molar quantity to the second input molar quantity is in a range of 1.0:1.0 to 1.0:1.1.

8. A method of claim 1, wherein the phase separation is performed in a phase separation tank having a temperature controlled in a range of 40 to 50° C.

9. An apparatus for preparing an ester, comprising:
a reactive distillation column having a middle section, and fed with an acid and an alcohol for a reaction to generate a first gas mixture;
a phase separation tank performing a phase separation for the first gas mixture for to generate a first liquid mixture, and transporting the first liquid mixture back to the reactive distillation column; and
a separative distillation column having a bottom section, and inputted with a second liquid mixture from the middle section of the reactive distillation column for performing a distillation separation therefor, wherein the ester is obtained at the bottom section of the separative distillation column.

10. An apparatus of claim 9, wherein:
the reactive distillation column has a bottom section and includes:
a first input port disposed at the bottom section of the reactive distillation column; and
a second input port disposed at the bottom section of the reactive distillation column, and
the apparatus further includes:
a first supply tank storing the acid;
a second supply tank storing the alcohol;
a first pipe connecting the first supply tank and the first input port;
a second pipe connecting the second supply tank and the second input port;
a first valve configured in the first pipe; and
a second valve configured in the second pipe, wherein the acid is transported from the first supply tank into the first pipe, via the first valve and the first input port, and then into the reactive distillation column, and the alcohol is transported from the second supply tank into the second pipe, via the second valve and the second input port, and then into the reactive distillation column.

11. An apparatus of claim 10, further comprising a catalyst disposed inside the reactive distillation column for accelerating the reaction, wherein the catalyst includes an ionic exchange resin, and the reaction includes an esterification reaction.

12. An apparatus of claim 10, wherein the acid includes an acetic acid, the alcohol includes one selected from a group consisting of an ethanol, an n-propanol, an isopropanol and a butanol, the acid has a first input molar quantity, the alcohol has a second input molar quantity, the apparatus has a molar ratio of the first input molar quantity to the second input molar quantity, and the molar ratio is in a range of 1.0:1.0 to 1.0:1.1.

13. An apparatus of claim 10, wherein:
the reactive distillation column has a top section and includes:
a first output port disposed at the top section of the reactive distillation column; and
a fourth input port disposed at the top section of the reactive distillation column;
the phase separation tank has a bottom section and includes:
a second output port; and
a third input port; and
the apparatus further includes:
a third pipe connecting the first output port and the third input port; and
a fourth pipe connecting the second output port and the fourth input port, wherein the first gas mixture is transported from the first output port, into the third pipe, via the third input port, and into the phase separation tank, the first liquid mixture is transported from the second output port, into the fourth pipe, via the fourth input, and into the reactive distillation column, and the apparatus has a by-product obtained at the bottom section of the phase separation tank.

14. An apparatus of claim 13, further comprising a first pump, a third valve and a fourth valve, wherein the first gas mixture is transported from the first output port, into the third pipe, via the third valve and the third input port, and into the phase separation tank, and the first liquid mixture is transported from the second output port, into the fourth pipe, via the first pump, the fourth valve and the fourth input port, and then into the reactive distillation column.

15. An apparatus of claim 13, wherein the phase separation tank has a bottom section, and the apparatus further comprises:
a third output port disposed in the bottom section of the phase separation tank;
a by-product tank;
a fifth pipe connecting the third output port and the by-product tank;
a second pump configured in the fifty pipe; and
a fifth valve configured in the fifty pipe, wherein the by-product is transported from the third output port, into the fifth pipe, via the second pump and the fifth valve, and then into the by-product tank, and the by-product is water.

16. An apparatus of claim 15, wherein:
the reactive distillation column includes:
a fourth output port disposed at the middle section of the reactive distillation column; and
a sixth input port disposed at the middle section of the reactive distillation column; and
the separative distillation column has a top section and includes:
a fifth input port disposed at the top section of the separative distillation column;
a fifth output port disposed at the top section of the separative distillation column; and
a second gas mixture generated at the top section of the separative distillation column, and transported from the fifth output port, via the sixth input port, and then into the reactive distillation column,
wherein the second liquid mixture is transported from the fourth output port, via the fifth input port, and then into the separative distillation column.

17. An apparatus of claim 16, wherein the reactive distillation column has a bottom section, and the apparatus further comprises:
a first reboiler:
a sixth pipe connecting the first reboiler and the bottom section of the reactive distillation column;
a seventh pipe connecting the first reboiler and the bottom section of the reactive distillation column; and
a third, liquid mixture generated at the bottom section of the reactive distillation column, transported to the first reboiler through the sixth pipe, and then transported from the first reboiler back to the reactive distillation column through the seventh pipe.

18. An apparatus of claim 17, further comprising:
a second reboiler;
a product tank;
an eighth pipe connecting the second reboiler and the bottom section of the separative distillation column;
a ninth pipe connecting the second reboiler and the bottom section of the separative distillation column; and
a fourth liquid mixture generated at the bottom section of the separative distillation column, transported to the second reboiler through the eighth pipe, and including:
a first portion transported from the second reboiler back to the separative distillation column through the ninth pipe; and
a second portion outputted from the second reboiler to the product tank.

19. An apparatus of claim 9, wherein the reactive distillation column has:
a first temperature controlled in a range of 60 to 130° C.;
a bottom section having a second temperature controlled in a range of 110 to 130° C.; and
a top section having a third temperature controlled in a range of 60 to 80° C.

20. An apparatus of claim 9, wherein the phase separation tank has a fourth temperature controlled in a range of 40 to 50° C., the separative distillation column has a top section having a fifth temperature controlled in a range of 55 to 65° C., and the bottom section of the separative distillation column has a sixth temperature controlled in a range of 75 to 85° C.
